(19) United States
(12) Patent Application Publication
(10) Pub. No.: US 2013/0302448 A1
(43) Pub. Date: Nov. 14, 2013

(54) PHYTIN AQUEOUS SOLUTION AND USE OF SAID SOLUTION FOR THE PREPARATION OF A LIQUID NUTRITIONAL SUPPLEMENT OR A COSMETIC COMPOSITION

(71) Applicant: Guy PARC, Rueil-Malmaison (FR)

(72) Inventor: Guy PARC, Rueil-Malmaison (FR)

(21) Appl. No.: 13/468,180

(22) Filed: May 10, 2012

(51) Int. Cl. A61K 31/6615; A23L 1/015; A23L 1/302; A23L 1/304; A23L 1/305; A61P 3/02; A61K 38/00; A61K 36/00; A61K 38/22; A61K 8/55; A61P 35/00; A23L 1/30; A23L 1/28

(52) U.S. Cl. 424/725; 426/648; 426/72; 426/74; 426/2; 514/102; 514/5.5; 514/9.7

(57) ABSTRACT

A phytin aqueous solution including at least one alpha-hydroxylated carboxylic acid and phytin in a solubilized form, preferably in a concentration ranging from 70 g/L to 270 g/L, and in that it has a pH value ranging from 3 to 5. The phytin aqueous solution may be used in the preparation of a liquid nutritional supplement and in the preparation of a cosmetic composition.

### FIELD OF THE INVENTION

The present invention relates to the field of phytin aqueous solution formulations and their applications in pharmaceutics, cosmetics and food industry.

### STATE OF THE ART

Phytic acid (CAS: 83-86-3), also known as myo-inositol hexakisphosphate, is a biomolecule that is present in oligoginous fruits and in the seeds of many cereal plants and legumes, wherein it represents up to 90% of phosphorous total storage. Phytic acid mainly occurs in the aleurone layer or in the seed embryo, wherein it comes either in the free acid form, or as phytates associated with different cations.

Phytic acid may be extracted from plant matrices in the form of phytin. As used herein, <<phytin>> means phytic acid salts (or phytates) which comprise essentially Mg²⁺, Ca²⁺ and/or K⁺ as counter-ions.

Phytic acid has long been considered as an antinutritional factor in human feed because it causes the absorption of some essential minerals such as calcium, magnesium and iron to be limited at the gastrointestinal tract level.

However, recent studies demonstrated that phytic acid and phytin may exert a wide range of benefits on human health (For review: Bohn and al., Daynal of Zhejang university Science B, 2008. 9(3):165-191).

Phytic acid and phytin may thus take part to the reduction of cholesterol levels, to the normalization of glycemia and to the stimulation of the immune system. Such compounds may also be used to prevent renal calculi, skin hyperpigmentation, and premature cell ageing as well, thanks to their antioxidizing activity.

It could also be shown that phytic acid can inhibit the main processes involved in carcinogenesis such as abnormal cell proliferation and angiogenesis. Phytic acid may also induce apoptosis and take part to the stimulation of the cell-mediated immune response. As a consequence, phytic acid and phytin are considered as being particularly interesting agents for cancer treatment but also for cancer prevention.

It appears therefore that a regular and controlled dietary intake of phytic acid or phytin may prevent the occurrence of a great number of diseases.

In the world's industrialized and developed countries, the average intake of phytic acid provided by food does range from 300 to 1200 mg per day depending on the dietary pattern, whereas the recommended daily intake for a satisfactory nutritional balance accounts for about 2500 mg per day.

To fully take advantage of the benefits of phytic acid and phytin on health, it is necessary to supplement phytic acid or phytin intake originating from food with nutritional supplements to be preferably regularly ingested.

Phytin-enriched nutritional supplements that are marketed nowadays are encapsulated or tableted compounds containing phytin in the form of a water-insoluble powder. This type of formulation is particularly unsuited to the elderly suffering from deglutition difficulties and also to old people and young children because of the risk of suffocation and false route.

It is possible to obtain phytic acid aqueous solutions. However, phytic acid has a high acidity and a very low stability in aqueous solution, which makes the use thereof unsuitable for a nutritional supplement.

Therefore, there is a need today for new liquid phytin-based formulations which are adapted, amongst others, to the preparation of nutritional supplements aimed at a wide variety of consumers.

### SUMMARY OF THE INVENTION

It is an object of the present invention to provide a phytin aqueous solution characterized in that:
it comprises at least one alpha-hydroxylated carboxylic acid,
it comprises phytin in a solubilized form, preferably in a concentration ranging from 70 g/L to 270 g/L and
its pH value does range from 3 to 5.

It is also an object of the present invention to provide a method for preparing a liquid nutritional supplement comprising phytin, characterized in that it comprises a step (i) consisting in combining (a) phytin, (b) at least one alpha-hydroxylated carboxylic acid and (c) an aqueous solution so as to obtain a solubilized phytin aqueous solution with a pH value of from 3 to 5.

It is a further object of the present invention to provide a phytin-based nutritional supplement characterized in that
(i) it consists in an aqueous solution with a pH value of from 3 to 7.5 preferably of from 3 to 5 and
(ii) it comprises:
   phytin in a solubilized form together with at least one alpha-hydroxylated carboxylic acid, said phytin being present in a concentration ranging from 70 g/L to 270 g/L, and
   optionally, at least one additional compound selected in the group consisting of minerals, vitamins and precursors thereof, amino acids and derivatives thereof, oligopeptides, fatty acids, hormones, flavonoids, plant extracts, myo-inositol and their combinations.

### DETAILED DESCRIPTION OF THE INVENTION

1. The Phytin Aqueous Solution of the Invention

The applicant demonstrated that it is possible to solubilize phytin in an aqueous medium by combining the same with at least one alpha-hydroxylated carboxylic acid. Said alpha-hydroxylated carboxylic acid acts both as a solubilizing agent and as a stabilizing agent for phytin. The alpha-hydroxylated carboxylic acid does, at least partially, prevent phytin's phosphate groups from hydrolyzing. The applicant could moreover observe that phytin solutions solubilized in the presence of alpha-hydroxylated carboxylic acid are more stable than traditional phytic acid aqueous solutions which must be stored at low temperature so as to limit hydrolyzing reactions.

Without wishing to be bound by any theory, the applicant is of the opinion that the stabilizing and solubilizing properties of the alpha-hydroxylated carboxylic acids towards phytin result from their weak-acid character and also from their chelating property due to the presence of both hydroxyl and carboxyl functional groups.

Surprisingly, the applicant showed that alpha-hydroxylated carboxylic acids are more efficient than non alpha-hydroxylated carboxylic acids for promoting both solubilization and stabilization of phytin in aqueous solutions and for significantly limiting the hydrolysis of the phosphate groups.

The applicant thinks that associating phytin with an alpha-hydroxylated carboxylic acid in aqueous solution causes soluble and highly stable supramolecular entities to be generated due to ionic bonds and hydrogen bonds between the phytate phosphate groups, to the divalent cations that are initially present in phytin and to the hydroxyl and carboxyl functional groups in the alpha-hydroxylated carboxylic acid.

As a consequence, the solution resulting from the combination of phytin with at least one alpha-hydroxylated carboxylic acid has a high solubility over time. Furthermore, as a characteristic, this solution has a pH value ranging from 3 to 5 which makes it suitable, amongst others, for use in cosmetic products and nutritional supplements.

Thus, it is a first object of the invention to provide a phytin aqueous solution characterized in that:
it comprises at least one alpha-hydroxylated carboxylic acid,
it comprises phytin in a solubilized form and
its pH value does range from 3 to 5.

Because phytin is in a solubilized form, the aqueous solution of the invention is clear. As used herein, a <<clear solution>> is intended to mean a solution wherein no turbidity or precipitate can be observed by naked eye.

As used herein, <<phytin>> is intended to mean phytic acid salts (also called phytates) comprising essentially Ca²⁺, Mg²⁺ and K⁺ as counter-ions. In other words, phytin corresponds to the phytic acid basic forms mainly associated with Ca²⁺ and/or Mg²⁺ and/or K⁺ cations. Phytin may comprise other counter-ions such as Na⁺, Fe²⁺, Fe³⁺, Co²⁺, Zn²⁺ and Cu²⁺.

Phytin may be obtained from cereal seeds. To be mentioned as plant sources suitable for preparing a phytin according to the invention wheat, corn and rice. As an example, phytin may be obtained from wheat bran, rice bran and corn seed embryo.

In some embodiments, phytin is used, which is obtained from cereal seeds and which has a calcium content, in weight percent, of from 0.05% to 20% and a magnesium content, in weight percent, of from 3% to 18%, the percentages being expressed relative to the phytin total weight.

As used herein, an <<aqueous solution>> is intended to mean a solution comprising water as main solvent, that is to say a solution comprising at least 90% by volume of water, in relation to the solution total volume. This aqueous solution may comprise from 0.1% to 10% by volume of a water-miscible solvent. To be mentioned as a water-miscible solvent to be suitably used for preparing a nutritional supplement is ethanol.

In some embodiments, the aqueous solution only contains water as a solvent.

A pH value ranging from 3 to 5 includes a pH value of 3, a pH value of 3.5, a pH value of 4, a pH value of 4.5 and a pH value of 5.

As used herein, <<at least one>> is intended to mean one or more. As an example, <<at least one alpha-hydroxylated carboxylic acid>> includes 1, 2, 3, 4, 5, 6, 7, 8, 9, 10 alpha-hydroxylated carboxylic acids.

As used herein, an <<alpha-hydroxylated carboxylic acid>> is intended to mean a chemical molecule comprising at least one carboxylic acid functional group and at least one hydroxyl functional group located onto the alpha carbon of said carboxylic acid functional group.

Preferably, alpha-hydroxylated carboxylic acids have following formula (I):

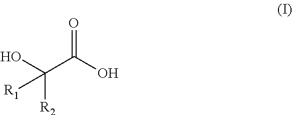

wherein:
   R1 and R2 are independently selected from hydrogen and linear or branched, C₁-C₁₄ alkyl groups, unsubstituted or substituted with one or more groups selected from carboxylic acid and hydroxyl groups, or
   R1 and R2 form a cyclic group selected from C₅-C₈ cycloalkyl and C₄-C₇ heterocycloalkyl groups, said cyclic group being unsubstituted or substituted with one or more groups independently selected from carboxylic acid and hydroxyl groups.

In the context of the present invention, a cycloalkyl group includes, amongst others, cyclopentyl, cyclohexyl and cycloheptyl groups.

In the context of the present invention, a C₄-C₇ heterocycloalkyl group includes, as a heteroatom, an oxygen or a nitrogen atom. To be mentioned as an illustrative heterocyclic alpha-hydroxylated carboxylic acid is glucuronic acid.

In some embodiments, the alpha-hydroxylated carboxylic acid is selected from compounds of formula I, wherein:
R1 is hydrogen and
R2 is a linear or branched C₁-C₁₀ alkyl group, unsubstituted or substituted with one or more groups independently selected from carboxylic acid and hydroxyl groups.

A C₁-C₁₀ group alkyl includes C₁, C₂, C₃, C₄, C₅, C₆, C₇, C₈, C₉ and C₁₀ alkyl groups.

Preferably, R2 is a C₁-C₅ alkyl group. C₁-C₅ alkyl radicals include methyl, ethyl, propyl, butyl and pentyl radicals.

It goes without saying that the alpha-hydroxylated carboxylic acid to be used is chosen depending on the intended application of the phytin solution of the invention. As an example, should the phytin solution of the invention be intended for use in the preparation of a nutritional supplement or a cosmetic product, the alpha-hydroxylated carboxylic acid must have a low toxicity and, if possible, a low allergenic effect.

In some embodiments, the alpha-hydroxylated carboxylic acid comprises at least one carboxylic acid group with a pKa ranging from 2.9 to 4.0.

The alpha-hydroxylated carboxylic acid may be selected from food additives, excipients suitable for use in the preparation of nutritional supplements, pharmaceutical compositions or cosmetic compositions.

In a preferred embodiment, the alpha-hydroxylated carboxylic acid is selected in the group consisting of glycolic acid, gluconic acid, glucuronic acid, diastereoisomers of tartaric acid, lactic acid, citric acid, malic acid and their combinations.

Said diastereoisomers of tartaric acid include meso-tartaric acid, levo-tartaric acid and dextro-tartaric acid.

The applicant showed that alpha-hydroxylated carboxylic acids enable to significantly increase the solubility of phytin in water. Thus, it is therefore possible to obtain solutions comprising a high content of phytin in the solubilized state. In a preferred embodiment, the phytin content does range from 70 g/L to 270 g/L, the phytin being in solubilized state and the phytin content being given relative to the phytin solution total volume.

A phytin content ranging from 70 g/L to 270 g/L includes a phytin content of 70 g/L, 80 g/L, 90 g/L, 100 g/L, 110 g/L, 120 g/L, 130 g/L, 140 g/L, 150 g/L, 160 g/L, 170 g/L, 180 g/L, 190 g/L, 200 g/L, 210 g/L, 220 g/L, 230 g/L, 240 g/L, 250 g/L, 260 g/L and 270 g/L.

To obtain optimum solubilization and stabilization of phytin, the applicant revealed that the amount of alpha-hydroxylated carboxylic acid to be provided should correspond to a carboxyl functional group proportion ranging from 3 mmol to 9 mmol per gram of phytin. The amount of alpha-hydroxylated carboxylic acid to provide so as to obtain a phytin stable solution does thus vary depending on the number of carboxyl groups present on said alpha-hydroxylated carboxylic acid. Thus, a carboxyl functional group proportion of from 3 mmol to 9 mmol per gram of phytin does correspond, for example to:
an amount of alpha-hydroxylated carboxylic acid with only one carboxyl group ranging from 3.0 mmol to 9.0 mmol per gram of phytin,
an amount of alpha-hydroxylated carboxylic acid with two carboxyl groups ranging from 1.5 mmol to 4.5 mmol per gram of phytin,
an amount of alpha-hydroxylated carboxylic acid with three carboxyl groups ranging from 1.0 mmol to 3.0 mmol per gram of phytin, or to
an amount of alpha-hydroxylated carboxylic acid with four carboxyl groups ranging from 0.75 mmol to 2.25 mmol per gram of phytin.

Thus, in some embodiments, the amount of alpha-hydroxylated carboxylic acid which is associated with phytin in a phytin solution of the invention does correspond to a carboxyl functional group proportion ranging from 3 mmol to 9 mmol per gram of phytin.

A carboxyl functional group proportion ranging from 3 mmol to 9 mmol includes a carboxyl functional group proportion of 3 mmol, 3.5 mmol, 4 mmol, 4.5 mmol, 5 mmol, 5.5 mmol, 6 mmol, 6.5 mmol, 7 mmol, 7.5 mmol, 8 mmol, 8.5 mmol and 9 mmol.

Preferably, the carboxyl functional group proportion does range from 4.5 mmol to 8 mmol per gram of phytin.

Based upon these data and depending on the final content of solubilized phytin the phytin solution of the invention should contain, the person skilled in the art can determine by routine experimentation the accurate amount of carboxyl functional group to be provided per gram of phytin and therefore, the amount of alpha-hydroxylated carboxylic acid to be used per gram of phytin.

For example, a solution containing 140 g/L of phytin may be obtained by providing an amount of citric acid of about 2 mmol per gram of phytin, which corresponds to a carboxyl functional group proportion of about 6 mmol per gram of phytin.

The phytin solution of the invention may be used in various types of applications, in particular in the pharmaceutical, food industry and cosmetic fields.

As an example, the phytin solution of the invention may be incorporated into cosmetic compositions such as creams, washing solutions, lotions and dental hygiene products.

Thus, it is a specific object of the present invention to provide the use of a phytin aqueous solution, said phytin having been solubilized together with at least one alpha-hydroxylated carboxylic acid, for the preparation of a cosmetic composition.

When incorporated into creams or lotions, the phytin solution may act as a skin lightening agent, an agent for preventing skin hyperpigmentation, EDTA substitute, anti-wrinkle agent, anti-ageing agent or anti-oxidizing agent.

The phytin solution of the invention may also be incorporated into toothpastes or dental lotions because of its effect on the dental plaque formation and on enamel.

The phytin solution may account for 0.2% to 95% by weight of the total weight of the relevant cosmetic composition. As regards the preparation of such cosmetic compositions, the person skilled in the art can refer to the reference books in the field, in particular to <<International Cosmetic Ingredient Dictionary and Handbook, 8th Ed., 2000 published by The Cosmetic, Toiletry and Fragrance Association.

The phytin solution of the invention may be advantageously used for the preparation of nutritional supplements.

This specific application will be detailed hereafter.

2. Liquid Nutritional Supplement and Method for Preparing Said Nutritional Supplement.

a. A Method for Preparing a Liquid Nutritional Supplement of the Invention

Without wishing to be bound by any theory, it is the opinion of the applicant that the phytin solutions of the invention have a bioavailability in phytin that is higher than in the phytin solid formulations described in the state of the art. As a consequence, the phytin aqueous solution of the invention is particularly suitable for use as a nutritional supplement or for the preparation of a nutritional supplement.

Thus, this is a second object of the present invention to provide a method for preparing a liquid nutritional supplement comprising phytin, characterized in that it comprises a step (i) consisting in preparing a phytin solution such as described in depth in the first part of the present specification entitled <<The phytin aqueous solution of the invention>>.

Said otherwise, the method for making the phytin-containing liquid nutritional supplement is characterized in that it comprises a step (i) consisting in combining (a) phytin, (b) at least one alpha-hydroxylated carboxylic acid and (c) an aqueous solvent so as to obtain an aqueous solution with a pH value of from 3 to 5 and comprising phytin in solubilized state.

Embodiments of the method of the invention as regards the characteristics of step (i) are directly derived from the elements described in part 1 entitled <<The phytin aqueous solution of the invention>>.

In particular, the phytin final content in the nutritional supplement does preferably range from 70 g/L to 270 g/L, phytin being in solubilized state and the content thereof being expressed in relation to the nutritional supplement total volume.

Preferably, the alpha-hydroxylated carboxylic acid used in step (i) does correspond to the hereabove chemical formula (I). As previously explained, to obtain an optimum solubilization and stabilization of phytin, an amount of alpha-hydroxylated carboxylic acid should be provided in step (i), which corresponds to a carboxyl functional group proportion ranging from 3 mmol to 9 mmol per gram of phytin.

As used herein, an <<aqueous solvent>> is intended to mean a liquid mainly consisted of water, that is to say comprising at least 90% of water by volume. An aqueous solvent may therefore comprise from 0.1% to 10% by volume of a water-miscible solvent. Ethanol is to be mentioned as water-miscible solvent suitable for preparing a nutritional supplement.

In some embodiments, the aqueous solvent is water.

In further embodiments, step (i) of the method of the invention comprises the sequence of following steps consisting in:
(a) preparing a phytin suspension in aqueous solution,
(b) adding, under stirring, to the suspension obtained in step (a), an amount of at least one alpha-hydroxylated carboxylic acid corresponding to a carboxyl functional group proportion ranging from 3 mmol to 9 mmol per gram of phytin, and
(c) keeping under stirring the phytin suspension enriched with the alpha-hydroxylated carboxylic acid obtained in step (b) until a clear solution is obtained.

As used herein, a <<clear solution>> is intended to mean a solution wherein neither turbidity nor precipitate can be observed by naked eye.

It is preferred in step (b) to add the alpha-hydroxylated carboxylic acid gradually or in small portions and to set the reaction medium under stirring so as to avoid the mass setting.

The alpha-hydroxylated carboxylic acid may be introduced in the form of a concentrated solution. As used herein, an <<alpha-hydroxylated carboxylic acid concentrated solution>> is intended to mean a solution with a concentration of at least 0.3 mol·l⁻¹. To ensure a complete solubilization of phytin, it may be necessary to keep the mixture of step (b) under stirring. The reaction being exothermal, it is generally not necessary to heat the reaction medium to achieve a complete solubilization of phytin.

The nutritional supplement obtained by implementing the method of the invention may comprise at least one additional compound having a nutritional, a physiological or a pharmaceutical benefit. It may be a compound that is able to interact with phytin, for example, a compound capable of stimulating the immune response-mediated defense or a compound having an action which is different from that of phytin. Advantageously, such additional compound may be a nutrient essential for human health, that is to say a nutrient that cannot be synthesized by the human body.

Thus, in some embodiments, the method of the invention comprises step (ii) consisting in adding to the solution obtained in step (i) at least one additional compound selected in the group consisting of minerals, vitamins and precursors thereof, amino acids, amino acid derivatives, oligopeptides, fatty acids, hormones, flavonoids, myo-inositol, plant extracts and their combinations.

In the context of the present invention, the minerals include the whole inorganic substances described in the state of the art as being essential for a good performance of the human body. Minerals include calcium, magnesium, iodine, iron, copper, zinc, selenium and manganese, this list being non exhaustive.

Vitamins include water-soluble vitamins such as group B vitamins, vitamin C and fat-soluble vitamins such as vitamins A, D, E and K.

As a rule, <<vitamins>> include a group of compounds. As used herein, vitamin E is thus intended to mean the group of the naturally occurring forms of vitamin E that is to say alpha-tocopherol, beta-tocopherol, gamma-tocopherol, delta-tocopherol, alpha-tocotrienol, beta-tocotrienol, gamma-tocotrienol and delta-tocotrienol. Similarly, vitamin D indifferently means ergocalciferol and cholecalciferol.

As used herein, vitamin precursors include those compounds involved in the biosynthesis cycle of vitamins. As an example, 7-dehydrocholesterol is a precursor of vitamin D.

In the context of the present invention, an <<amino acid>> is intended to mean a chemical molecule comprising a primary amine function in alpha position of a carboxylic acid group. Thus, an <<amino acid>> includes natural amino acids, in particular amino acids from the genetic code and synthetic amino acids. Preferably, the amino acid is selected from basic amino acids such as L-histidine, L-ornithine, L-lysine and L-arginine.

In the context of the present invention, oligopeptides comprise from 2 to 50 amino acids, preferably from 2 to 10 amino acids. Preferably, the oligopeptide comprises at least one amino acid selected from L-histidine, L-ornithine, L-lysine and L-arginine.

By “amino acid” is also encompassed various chemical states of the acid such as salified, hydrate or solvate forms of said amino-acid.

In some embodiments of the composition according to the invention, the molar ratios between the equivalent phytic acid of the phytin and the non-peptide amine, guanidine or imidazole groups of the basic compounds defined above is ranging from 1/12 to 1 and in particular is between ⅙ and 1/9.

Taurine can be mentioned as an example of amino acid derivatives.

Fatty acids comprise, in particular, the omega 3 family. As a non-limiting illustration are to be mentioned as fatty acids belonging to the omega 3 family, α-linolenic acid, eicosapentaenoic acid and docosahexaenoic acid.

Flavonoids include plant biomolecules comprising a flavone central unit. To be mentioned as suitable examples are quercetin and hesperidin.

Myo-inositol represents the dephosphorylated form of phytic acid and corresponds to the compound with CAS No: 87-89-8.

To be mentioned as suitable hormone examples are melatonin, DHEA and phyto-estrogenes.

Lastly, to be mentioned as suitable examples of plant extracts are ginseng, rhodiola, guarana and kola extracts.

The additional compound(s) to be added in step (ii) may be determined depending on the expected physiological effects or depending on the target users. As an example, should a high antioxidizing activity be expected, to phytin may be associated, for example, a compound selected amongst vitamin C, vitamin E and quercetin or even a green tea extract. Should the nutritional supplement be aimed at a consumer suffering from a nutritional imbalance, phytic acid may be combined with a cocktail of minerals and vitamins. If the physiological effect expected is to reinforce stress resistance, or to improve the intellectual concentration or the physical endurance, the nutritional supplement of the invention may comprise a plant extract such as a ginseng extract (Panax ginseng C. A. Meyer), a rhodiola extract (Rhodiola rosea L.), a guarana extract (Paullinia cupana Kunth) and a kola extract (Cola nitida alba, Cola nitida rubra A. Chev, or Cola acuminata Schott & Endl)).

The one or more additional compounds having a physiological, pharmaceutical or nutritional aim are typically present in the liquid nutritional supplement in a concentration ranging from 0.01 g/L to 900 g/L, preferably from 0.1 g/L to 400 g/L. The amount of additional compound is to be determined depending on the nature of such compound and on the expected aim. Since additional compounds are commonly used in the preparation of nutritional supplements and pharmaceutical compositions, the person skilled in the art may refer to the doses traditionally employed in the state of the art for determining, for each additional compound, the concentration in which it should be introduced into the nutritional supplement of the invention.

In a preferred embodiment according to the invention, in step (ii) of the method are added myo-inositol and a vitamin C-containing compound that is to say ascorbic acid or a salt thereof.

Without wishing to be bound by any theory, the applicant thinks that such a combination is particularly suitable for preventing cancers and stimulating the immune system. Indeed, it could be demonstrated that associating phytin with myo-inositol causes some biological processes involved in carcinogenesis to be influenced such as cell abnormal proliferation and angiogenesis.

Furthermore, it is expected that phytic acid and vitamin C act synergistically to stimulate the immune response and prevent the oxidizing stress and thus cellular aging.

Preferably, myo-inositol is introduced in step (ii) of the method according to the invention so that its final concentration in the nutritional supplement be within a range of from 3 g/L to 6 g/L. Ascorbic acid or a salt thereof is introduced so that its final concentration in the nutritional supplement be within a range of from 6 g/L to 12 g/L.

As already stated hereabove, the method for preparing a liquid nutritional supplement having a phytin content ranging from 70 g/L to 270 g/L according to the present invention comprises the steps consisting in:
(i) combining a phytin, at least one alpha-hydroxylated carboxylic acid and an aqueous solution so as to obtain a phytin aqueous solution with a pH value of from 3 to 5 and
(ii) optionally, adding to the solution obtained in step (i) at least one additional compound selected in the group consisting of minerals, vitamins and precursors thereof, amino acids and derivatives thereof, oligopeptides, plant extracts, fatty acids, hormones, flavonoids, myo-inositol, and their combinations. The method according to the present invention may comprise in addition, a step of filtrating the solution obtained in step (i) and/or in step (ii). This filtration step makes it possible to remove the possible suspended residual solid particles.

Lastly, the method according to the present invention may comprise a packaging final step. As an example, the nutritional supplement of the invention may be packaged in a multi-dose container or in unit-dose containers.

The production method according to the present invention may be implemented by using methods commonly used in the food industry and in the pharmaceutical industry. Particularly, it is necessary to implement the production method according to the present invention while respecting the laboratories good practices and the applicable standards. The production method of the invention should be carried out under aseptic conditions using raw materials with a purity adapted to their use in a nutritional supplement.

b. The Liquid Nutritional Supplement Enriched with Phytin of the Invention

It is a further object of the present invention to provide a phytin-containing liquid nutritional supplement which may be obtained from the production method described hereunder.

The liquid nutritional supplement of the invention is characterized in that:
(i) it is an aqueous solution with a pH value of from 3 to 7.5, preferably of from 3 to 5, and
(ii) it comprises phytin in a solubilized form together with at least one alpha-hydroxylated carboxylic acid, said phytin being present in a concentration ranging from 70 g/L to 270 g/L, the phytin content being expressed relative to the nutritional supplement total volume.
   As previously explained, an <<aqueous solution>> is intended to mean a solution comprising water as major solvent.

In some embodiments, the nutritional supplement of the invention may contain a small amount of a water-miscible solvent, for example from 0.5% to 10% by volume of a solvent such as ethanol, such percentage being expressed in relation to the nutritional supplement total volume. The percentage by volume of water in the nutritional supplement is then within the range of from 99.5% to 90%.

In other embodiments, water is the only one solvent present in the nutritional supplement according to the present invention.

As previously described, to ensure optimum solubilization and stabilization of phytin, the amount of alpha-hydroxylated carboxylic acid present in solution should preferably correspond to a carboxyl functional group amount ranging from 3 mmol to 9 mmol per gram of phytin.

In a preferred embodiment, the alpha-hydroxylated carboxylic acid is selected from citric acid, lactic acid, tartaric acid, malic acid and their combinations.

The liquid nutritional supplement may comprise at least one additional compound having a nutritional, physiological or pharmaceutical aim.

Thus, the liquid nutritional supplement comprising phytin according to the present invention is characterized in that:
(i) it is an aqueous solution with a pH value of from 3 to 7.5, preferably of from 3 to 5.
(ii) it comprises phytin in a solubilized form together with at least one alpha-hydroxylated carboxylic acid, said phytin being present in a concentration ranging from 70 g/L to 270 g/L, the phytin content being expressed relative to the nutritional supplement total volume and
(iii) optionally, at least one additional compound selected in the group consisting of minerals, vitamins and precursors thereof, amino acids, amino acid derivatives, oligopeptides, plant extracts, fatty acids, hormones, flavonoids, myo-inositol, and their combinations. As previously explained, the additional compound may either reinforce the phytin benefits, or generate a physiological effect different from that of the phytin.

Building on her/his general knowledge in physiology and in pharmacy, the person skilled in the art will be able to select the one or more additional compounds to be added depending upon (i) the target consumer and (ii) the expected physiological effect.

Thus, if at the same time (i) an antioxidizing action, (ii) a prophylactic action towards cancer and (iii) a stimulation of the immune system are expected, a liquid nutritional supplement will be advantageously formulated comprising both phytin, a vitamin C-containing compound and myo-inositol.

Furthermore, if the antineoplastic effect of phytin is to be potentiated, the nutritional supplement may advantageously comprise at least one compound selected from basic amino acids such as L-lysine, L-histidine, L-arginine and L-ornithine and oligopeptides comprising at least one basic amino acid within their structure. For example, the nutritional supplement may comprise soy peptone. Soy peptones contain more arginine or lysine in the free state or in the form of oligopeptides depending on their degree of hydrolysis.

The one or more additional compounds having a physiological, pharmaceutical or nutritional aim are typically present in the liquid nutritional supplement in a concentration ranging from 0.01 g/L to 900 g/L, preferably from 0.1 g/L to 400 g/L. The amount of additional compound is to be determined depending on the nature of such compound and on the expected aim. The person skilled in the art may refer to the doses commonly used in the state of the art to determine, for each additional compound, the concentration in which said compound should be introduced into the nutritional supplement according to the present invention.

In a particular embodiment, the nutritional supplement of the invention further comprises myo-inositol in a concentration ranging preferably from 3 g/L to 6 g/L and ascorbic acid or a salt thereof in a concentration ranging from 6 g/L to 12 g/L.

Daily doses of phytin and compounds having a nutritional, physiological or pharmaceutical aim optionally present in the nutritional supplement may be determined considering, amongst others, daily nutritional intakes recommended in the state of the art. The nutritional supplement dosage can also be adjusted depending on the physiological effect expected and depending on the physiological condition of the target recipient of the nutritional supplement. In particular, individual factors will be taken into account, such as age, weight, sex, general condition and diet of the target consumer of the liquid nutritional supplement.

As regards phytin, a daily nutritional intake of at least 2500 mg is recommended whereas the dietary pattern followed in the industrialized countries provides 300 mg to 1200 mg per day of phytin. Thus, the phytin daily dose to provide to consumers does preferably range from 1300 mg to 2200 mg.

The liquid nutritional supplement of the invention may further comprise one or more excipients selected from pharmaceutical excipients or food additives.

To be mentioned as suitable examples of pharmaceutical excipients and food additives, are texturizing agents, emulsifying agents, preservatives, coloring agents, natural or synthetic flavoring agents, sweeteners and flavor enhancers.

In some embodiments, the liquid nutritional supplement of the invention comprises at least one excipient selected from preservatives, coloring agents, flavoring agents, sweeteners and flavor enhancers.

These compounds are intended to make more pleasant the ingestion of the liquid nutritional supplement for the consumer. This is particularly important when the target consumer is a child. As an example, to facilitate and make more pleasant the ingestion of the nutritional supplement, to the nutritional supplement of the invention may be added:
(i) a citrus-like flavor
(ii) a coloring agent providing a yellow-orange color
(iii) a sweetener
so as to call a sensation of fruit juice to mind when taking said nutritional supplement.

As previously stated, the nutritional supplement of the invention is highly stable over time. Therefore, a preservative or a stabilizer not necessarily needs to be added to the nutritional supplement when packaged in unit-doses.

Thus, in some embodiments, the liquid nutritional supplement does not comprise any preservative or stabilizer other than the alpha-hydroxylated carboxylic acid. As an illustration, sodium sorbate, potassium benzoate, acetic acid and sodium nitrite may be mentioned as preservatives, the list being non exhaustive.

When the nutritional supplement is packaged in multi-doses, it is preferred to add a preservative so as to avoid any bacterial or fungal contamination.

In other embodiments, the nutritional supplement is free of any texturizing agent such as thickeners and gelling agents. Indeed, the presence of these agents may be detrimental to the liquid property and to the homogeneity of the nutritional supplement. In particular, the nutritional supplement does not contain any gelling compound such as agar-agar, starch, alginate salt, carrageenan and animal gelatins.

The nutritional supplement of the invention may be packaged in multi-dose or single-dose containers. Preferably, the nutritional supplement is packaged in dose units, for example in the form of single-dose vials or single-dose easy-break ampules.

The liquid nutritional supplement of the invention may be administered for completing phytin intakes derived from food. In other words, the nutritional supplement of the invention, amongst others, is intended to ensure to the consumer a regular and sufficient phytin daily intake. Such an intake makes it possible to maintain or to improve the health general condition of the consumer who takes the nutritional supplement of the invention.

It may also be prescribed to a consumer suffering from a physiological disorder or capable of developing a physiological disorder or a disease. In particular, the nutritional supplement of the invention may be prescribed to prevent renal calculi or cellular aging, to normalize the cholesterol level or the blood sugar level, to stimulate the immune system or prevent the occurrence of cancer. The nutritional supplement of the invention may also be prescribed to improve stress resistance, intellectual concentration and physical endurance.

It has been shown that the nutritional supplement of the invention has excellent anti-proliferative effect on various cancer cell lines, and is therefore useful for preventing or alleviating conditions related to hyper proliferation of the body cells, in particular tumoral disorders and cancers.

The invention is therefore also directed to methods for substantially inhibiting and/or limiting the development of cancer, comprising providing to a subject a nutritional supplement or a phytin aqueous solution according to the various embodiments disclosed in the instant specification.

The nutritional supplement of the invention may also be used for cosmetic purposes for maintaining or improving the skin general condition by preventing the aging thereof and the occurrence of hyperpigmentation spots.

It may also be used in order to enhance the therapeutic effect of a treatment or to improve the general condition of a patient following a hard therapeutic treatment, having been submitted to a surgical treatment and/or being unable to eat normally. It may be advantageous, in particular, to prescribe the nutritional supplement of the invention to patients following chemotherapeutic or radiotherapeutic treatments.

In particular, the phytin solutions or nutritional supplements useful for the above applications may comprise at least citric acid as alpha-hydroxylated carboxylic acid, and at least a basic amino acid, said amino acid being present either under the form of a monomer and/or in included in an oligopeptide as defined above.

The basic amino acid may in particular be a lysine, notably lysine hydrate.

The concentrations of the basic amino acids in the nutritional supplement will be adapted by the man skilled in the art, according to the amino acid(s) and their form and/or chemical state.

For instance, the concentrations of L-lysine hydrate can range from 20 g/l to 300 g/L, in particular from 50 g/L and 150 g/L.

The phytin-based liquid nutritional supplement of the present invention has many advantages as compared to the nutritional supplements available on the market, which as far as the applicant knows, consist in capsules or tablets containing powdered phytin.

Due to its liquid form, the nutritional supplement can be administered indifferently to children, to elderly persons and to any individual who encounters swallowing problems. It is expected that the therapeutic observance will be adopted by the consumers thanks to the liquid form formulation.

Lastly, as previously mentioned, it is expected that phytin present in the nutritional supplement of the invention, which is in a solubilized form in the presence of alpha-hydroxylated carboxylic acid, has a higher bioavailability than phytin in the form of capsules or tablets.

Use of a phytin aqueous solution is a further object of the present invention, said phytin having been solubilized together with at least one alpha-hydroxylated carboxylic acid, for the preparation of a nutritional supplement.

The present invention, without being limited thereto, will be illustrated through the following examples.

### EXAMPLES

### Example 1

### a. Solubilization of Phytin Through Carboxylic Acids

For comparison purposes of the solubilizing capacity of the various carboxylic acids with each other, the following procedure has been carried out:

For each carboxylic acid, an aqueous solution of normality 0.3 mol·l⁻¹ was prepared.

As a reminder, the normality of an acid solution does correspond to the number of moles of protons H⁺ provided by 1 liter of this solution. Thus, a citric acid solution with a normality of 0.3 N does correspond to a citric acid solution having a molarity of 0.1 mol·l⁻¹ because citric acid comprises 3 carboxylic acid groups. Said otherwise, 1 mole of citric acid may provide 3 moles of protons H.

A phytin suspension was prepared by suspending 1 g of phytin in a volume of 10 ml of distilled water. The phytin used is the one marketed by Tsuno Rice Fine Chemicals Co., Ltd under the reference PHYTIN (RICE BRAN EXTRACT). Its composition is given in Table 1 hereunder. It should be noted that this phytin is highly concentrated in magnesium.

TABLE 1
Phytin composition used
Phytin highly concentrated in magnesium marketed by
Tsuno Rice Fine Chemicals Co., Ltd extracted from rice
bran (Wakayama, Japan)

| | |
|---|---|
| Purity | 99.7% |
| Phosphorus content | 20% |
| Magnesium content | 12.7% |
| Calcium content | 0.5% |
| Potassium content | 4.6% |

* the contents correspond to weight percentages relative to the total weight of the commercially available phytin.

The 0.3N carboxylic acid solution was gradually added to the phytin suspension under stirring using a graduated buret according to a protocol equivalent to a pH-metric titration. The evolution of the pH value was followed by means of pH meter and the carboxylic acid solution volume required for the complete solubilization of phytin was determined.

Table 2 hereunder gives the results obtained for the various carboxylic acids tested.

It clearly appears that carboxylic acids having at least one hydroxyl radical in the alpha position of a carboxyl have a higher solubilizing capacity than non alpha-hydroxylated carboxylic acids. Indeed the amount of carboxyl functional groups to be provided for alpha-hydroxylated carboxylic acids is much lower than for non alpha-hydroxylated carboxylic acids. This fact is emphasized by the results obtained for maleic acid and succinic acid which both comprise 2 carboxylic acid groups.

The number of carboxyl groups carried by the alpha-hydroxylated carboxylic acid does not seem to directly influence its solubilization effect on phytin.

TABLE 2
Evaluation of the carboxylic acid solubilizing capacity towards
commercially available phytin extracted from rice bran

| | | |
|---|---|---|
| | | Volume added to |
| | | completely solubilize |
| Acid solution | Number of carboxyl groups | phytin |

Alpha-hydroxylated carboxylic acid

| | | | |
|---|---|---|---|
| | | | Amount of carboxyl |
| | | | functional groups |
| | | | provided |
| | | | (per gram of phytin) |
| Malic acid | 2, from which 1 with | 17.5 ml | 5.25 mmol |
| 0.15 mol · l⁻¹ | hydroxyl radical in alpha | | |
| Citric acid | 3, from which 1 with | 17.0 ml | 5.10 mmol |
| 0.1 mol · l⁻¹ | hydroxyl radical in alpha | | |
| Tartaric acid | 2, from which 2 with | 17.2 ml | 5.16 mmol |
| 0.15 mol · l⁻¹ | hydroxyl radical in alpha | | |

Non alpha-hydroxylated carboxylic acid

| | | | |
|---|---|---|---|
| | | | Amount of carboxyl |
| | | | functional groups |
| | | | provided |
| Acetic acid 0.3 mol · l⁻¹ | 1 | 35.5 ml | 10.65 mmol |
| Succinic acid 0.15 mol · l⁻¹ | 2 | 36.0 ml | 10.80 mmol |

### b. Influence of the Phytin-Based Composition

Experiments based on a protocol similar to that given in a. hereabove were conducted with a phytin available on the market highly concentrated in calcium also marketed by Tsuno Rice Fine Chemicals Co., Ltd (see Table 3 hereunder). Two carboxylic acid solutions 0.3 N were tested, i.e. citric acid and acetic acid.

Citric acid is very efficient to solubilize this form of phytin characterized by a high content of calcium. Indeed, a volume of 22.3 mL of citric acid solution 0.3 N (which corresponds to a proportion of carboxyl group of 6.7 mmol) made it possible to completely solubilize phytin and obtain a clear solution.

On the contrary, acetic acid revealed ineffective to fully solubilize the phytin. Indeed, even after adding a volume of 65 ml of acetic acid solution 0.3 N (i.e. an equivalent of 19.5 mmol of carboxyl groups), phytin was still incompletely dissolved as shown by the turbidity of the solution.

TABLE 3
Composition of the phytin with a high content of calcium marketed by
Tsuno Rice Fine Chemicals Co., Ltd
Commercially available phytin extracted from rice bran
highly concentrated in calcium and marketed by Tsuno
Rice Fine Chemicals Co., Ltd

| | |
|---|---|
| Purity | 99.7% |
| Phosphorus content | 20.7% |
| Magnesium content | 4.0% |
| Calcium content | 16.4% |

* the contents correspond to weight percentages relative to the total weight of the commercially available phytin.

### Example 2

### Method for Preparing a Nutritional Supplement Enriched with Phytin

Step 1: Preparation of a Stabilized Phytin Solution

The phytin used is a phytin highly concentrated in magnesium marketed by Tsuno Rice Fine Chemicals Co., Ltd, the composition of which is given in Table 1 hereabove.

21.1 kg of phytin were gradually added to 100 L of purified water under stirring. After homogenization, 25 L of an aqueous citric acid solution (1.68 mol·L⁻¹) were gradually introduced to the reaction medium. The reaction medium was maintained under stirring for about 45 minutes until a clear solution was obtained with a stabilized pH value.

The phytin solution obtained had a pH value of 4.1, a phytin content of 168.9 g/L and a concentration in citric acid of 0.34 mol/L.

Step 2: Addition of Compounds Having a Physiological Aim and Preparation of the Nutritional Supplement of the Invention

To 125 L of the solution obtained in step 1 were added, under stirring, 0.68 kg of myo-inositol and 1.20 kg of ascorbic acid. The volume of the resulting mixture was adjusted to 150 L. After homogenization, the mixture was filtered on a nylon filter with mesh openings of 50 μm.

The nutritional supplement obtained was an amber-colored clear solution, having a pH value of 4.0 and following composition:
140 g/L of phytin
0.28 mol/L of citric acid i.e. a carboxyl functional group amount equal to 6 mmol per gram of phytin
4.5 g/L of myo-inositol and
8 g/L of ascorbic acid

After the filtration step, the nutritional supplement was packaged in single-dose easy-break ampules of 10 mL. The nutritional supplement obtained could be stored several months away from direct light, at room temperature or in a refrigerator. As an alternative, the nutritional supplement could be packaged in vials of 200 ml fitted with a measuring cap. For this type of conditioning, the nutritional supplement should preferably comprise a preservative, be stored in a refrigerator and has to be preferably consumed within the 20 days after opening.

### Example 3

### Dosage of the Nutritional Supplement of the Invention

For an initial treatment intended to restore the physiological condition of the consumer or to induce a particular physiological effect (for example stimulating the immune system), the recommended dosage is one 15 mL-ampule of nutritional supplement per day which corresponds to a daily dose of phytin of 2100 mg. The daily dose of myo-inositol and ascorbic acid is 120 mg and 67.5 mg per day, respectively. Such a regimen has to be followed 8 days long in average and a couple of weeks at the most.

For a long-term treatment intended to maintain the physiological condition of the patient and to prevent the occurrence of some diseases, the recommended dosage is 10 mL of nutritional supplement per day, which corresponds to a dose of phytin of 1400 mg/day, a dose of ascorbic acid of 80 mg/day and a dose of myo-inositol of 45 mg/day.

The nutritional supplement may be taken at any time of the day. However, it is recommended to take the nutritional supplement of the invention in the morning on an empty stomach, diluted in some water or fruit juice.

### Example 4

### Antiproliferative Effect of the Dietary Supplement

a) The solution of dietary supplement described in Example 2, Step 2 was evaluated for its anti-proliferative effect on several cancer cell lines.
   Following strains were examined:
   DU 145: human prostatic cancer
   EMT 6: mouse mammary cancer
   SW 480: human colon adenocarcinoma

### Experimental Protocol

The cells were maintained in culture according to the following protocol:

### Culture Medium:

Cells were maintained in DMEM (Lonza) containing 10% decomplemented fetal calf serum (Dutscher)

### Culture

Culture flasks vented 75 cm² (Nunc) are used for the maintenance of lines in an oven at 37° C. with 5% carbon dioxide in the atmosphere saturated with moisture.
To test products, the cells are placed in culture wells of 96-well plate (Nunc) at a rate of 10,000 cells per well in 200 μl of culture medium. After overnight incubation, cultures were continued for 3 days in the presence of increasing concentrations of mixtures to be tested.
The measurement of cell viability is performed by a test XTT. The measurements are performed on equipment VICTOR 3V (Perkin Elmer Corporation).
The results are expressed as % viability

| Concentrations | DU 145 | EMT-6 | SW 480 |
|---|---|---|---|
| 0 mM | 100 | 100 | 100 |
| 0.0025 mM | 81.5 | 90.3 | 90.4 |
| 0.025 mM | 89.3 | 70.5 | 102.5 |
| 0.25 mM | 37.0 | 16.2 | 29.3 |
| 2.5 mM | 17.3 | 25.8 | 25.5 |

This table shows that the IC₅₀ of the three cancer cell lines of the solution of the food supplement described in Example 2, Step 2 is slightly less than 0.25 mM.
b) The same tests were conducted on the solution of the food supplement described in Example 2, Step 2 with the difference that the solution also contains 39.4 g/L of L-Lysine hydrate
   The results are substantially similar to those of the previous table except that, for strains EMT-6 and SW 340 and the maximum concentration of 2.5 mM, the growth of cancer cells is completely inhibited.

1. A method for preparing a liquid nutritional supplement comprising phytin, characterized in that it comprises a step (i) consisting in combining a phytin, at least one alpha-hydroxylated carboxylic acid and an aqueous solvent so as to obtain an aqueous solution comprising phytin in solubilized state, preferably in a concentration ranging from 70 g/L to 270 g/L, and with a pH value of from 3 to 5.

2. A preparation method according to claim 1, characterized in that the amount of alpha-hydroxylated carboxylic acid used in step (i) corresponds to a carboxyl functional group proportion ranging from 3 mmol to 9 mmol per gram of phytin.

3. A method for preparing a nutritional supplement according to claim 1, characterized in that the alpha-hydroxylated carboxylic acid is selected in the group consisting of lactic acid, diastereoisomers of tartaric acid, glycolic acid, gluconic acid, glucuronic acid, citric acid, malic acid and their combinations.

4. A method according to claim 1, characterized in that step (i) comprises the steps consisting in:
(a) preparing a phytin suspension in aqueous solution,
(b) adding to the suspension of step (a) an alpha-hydroxylated carboxylic acid, preferably as an aqueous concentrated solution.

5. A preparation method according to claim 1, characterized in that phytin is a phytin obtained from cereal seeds and has a calcium content, in weight percent, of from 0.05% to 20% and a magnesium content, in weight percent, of from 3% to 18%, the percentages being expressed relative to the phytin total weight.

6. A method for preparing a nutritional supplement according to claim 1, characterized in that it comprises step (ii) consisting in adding to the solution obtained in step (i) at least one additional compound selected in the group consisting of minerals, vitamins and precursors thereof, amino acids and derivatives thereof, oligopeptides, plant extracts, fatty acids, hormones, flavonoids, myo-inositol, and their combinations.

7. A method for preparing a nutritional supplement according to claim 6, characterized in that are added in step (ii):
ascorbic acid, or a salt thereof, so that the end concentration in ascorbic acid in the nutritional supplement be comprised between 6 g/L and 12 g/L, and
myo-inositol, so that the end concentration in myo-inositol in the nutritional supplement be comprised between 3 g/L and 6 g/L.

8. A method for preparing a liquid nutritional supplement according to claim 1, characterized in that it comprises a step of filtration.

9. A liquid nutritional supplement comprising phytin, characterized in that
(i) it is an aqueous solution with a pH value of from 3 to 7.5, preferably of from 3 to 5.
(ii) it comprises phytin in a solubilized form, together with at least one alpha-hydroxylated carboxylic acid, said phytin being present in a concentration ranging from 70 g/L to 270 g/L, the phytin content being expressed relative to the nutritional supplement total volume and
(iii) optionally, at least one additional compound selected in the group consisting of minerals, vitamins and precursors thereof, amino acids and derivatives thereof, oligopeptides, plant extracts, fatty acids, hormones, flavonoids, myo-inositol, and their combinations.

10. A nutritional supplement according to claim 9, characterized in that the content of alpha-hydroxylated carboxylic acid corresponds to a carboxyl functional group amount ranging from 3 mmol to 9 mmol per gram of phytin.

11. A nutritional supplement according to claim 9, characterized in that it contains at least an additional compound (iii) which is selected among basic amino acids and oligopeptides containing such amino acids.

12. A nutritional supplement according to claim 9, characterized in that it comprises:
myo-inositol in a concentration ranging from 3 g/L to 6 g/L, and
ascorbic acid, or a salt thereof, in a concentration ranging from 6 g/L to 12 g/L.

13. A nutritional supplement according to claim 12, characterized in that it contains at least a basic amino acid, said amino acid being under the form of a monomer and/or in an oligopeptide, and the molar ratio between the equivalent phytic acid of the phytin and the non-peptide amine, guanidine or imidazole groups of the basic amino acids is ranging from 1/12 to 1.

14. A phytin aqueous solution characterized in that:
it comprises at least one alpha-hydroxylated carboxylic acid,
it comprises phytin, in a solubilized form, in a concentration ranging from 70 g/L to 270 g/L and
its pH value does range from 3 to 5.

15. A phytin aqueous solution according to claim 14, characterized in that the amount of alpha-hydroxylated carboxylic acid corresponds to a carboxyl functional group proportion ranging from 3 mmol to 9 mmol per gram of phytin.

16. (canceled)

17. Method of substantially inhibiting and/or limiting the development of cancer, comprising providing to a subject a nutritional supplement according to claim 9.

18. Method of substantially inhibiting and/or limiting the development of cancer, comprising providing to a subject a phytin aqueous solution according to claim 14.

19. A nutritional supplement comprising the phytin aqueous solution of claim 12.

20. A cosmetic composition comprising the phytin aqueous solution of claim 12.
